(19) United States
(12) Patent Application Publication
(10) Pub. No.: US 2018/0021535 A1
(43) Pub. Date: Jan. 25, 2018

(54) HOSE FOR RESPIRATORY DEVICE

(71) Applicant: HANCOCK MEDICAL, INC., Mountain View, CA (US)

(72) Inventors: Thomas G. GOFF, Mountain View, CA (US); Kirby CHIANG, Mountain View, CA (US)

(73) Assignee: HANCOCK MEDICAL, INC., Mountain View, CA (US)

(21) Appl. No.: 15/551,671

(22) PCT Filed: Feb. 11, 2016
(86) PCT No.: PCT/US16/17531

§ 371 Date: Aug. 17, 2017

Related U.S. Application Data
(60) Provisional application No. 62/119,046, filed on Feb. 20, 2015.

(51) Int. Cl. A61M 16/06; A61M 16/08

(52) U.S. Cl. CPC A61M 16/0605; A61M 16/0875; A61M 16/0858; A61M 16/10

(57) ABSTRACT

Embodiments of a hose for use with a respiratory device are disclosed. The hose comprises a structural element and a sealing element. The sealing element can be inside or outside of the structural element. The sealing element and structural element are only selectively connected. Such a hose design provides greater flexibility and enhanced user comfort.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Respiratory devices such as CPAP systems use hoses to deliver pressurized air from a blower or other pressure source to a mask on the user's face. Such hoses are typically formed of a helix of stiff plastic thoroughly bonded to thinner plastic that extends between the helix coils. The stiff plastic provides crush resistance, while the thinner plastic provides the air seal. Prior examples of respiratory device hoses may be found in U.S. Pat. No. 8,453,681 and US Publication No. 2014/0053939.

One drawback of prior respiratory device hoses is their response to torsion. Because the helix is connected to the thin webbing, any torsion applied to one end of the hose (e.g., at the mask) can result in movement of the entire hose as the torsion force is transmitted down the helix and webbing. Current hoses exhibit near 1:1 torqueability, fully transferring any torsion from one end to the opposite end. Also, the ridges formed by the helix on the exterior of the hose can be noisy as they move across the ridges of bedside tables or bed frames when the user moves.

Finally, prior hoses are bulky and difficult to pack when traveling with a CPAP device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an embodiment of a hose for use with a respiratory device.

FIG. 2 illustrates the hose of FIG. 1 connected to a positive airway pressure device.

FIG. 3 illustrates an embodiment of a structural element of a hose.

FIG. 4 illustrates an embodiment of the hose with an internal liner.

FIG. 5 illustrates an embodiment of the hose with an internal liner and an outer layer.

FIGS. 6A-6H illustrate single wall, cross sectional views of embodiments of spiral structural elements of a hose.

FIGS. 7A-7C illustrate embodiments of tubes with various attachment configurations for the inner and outer layers.

FIG. 8 illustrates an embodiment of a structural element of a hose.

FIG. 9 illustrates an embodiment of a structural element of a hose.

FIG. 10 illustrates an embodiment of a structural element of a hose.

FIG. 11 illustrates an embodiment of a structural element of a hose.

FIG. 12 illustrates an embodiment of a structural element of a hose.

FIG. 13 illustrates a tubing connector of the prior art.

FIG. 14 illustrates an embodiment of a tubing connector.

FIG. 15 illustrates an embodiment of the hose showing an attachment pattern.

FIG. 16 illustrates an embodiment of the hose showing an attachment pattern.

FIG. 17 illustrates an embodiment of the hose showing an attachment pattern.

### DETAILED DESCRIPTION

Disclosed herein are hoses for use with respiratory devices (e.g., positive airway pressure therapy, ventilation, oxygen delivery, air delivery, gas delivery, medication delivery, smoke evacuation, and anesthesia applications). The hoses generally include a structural element and a sealing element. The structural element and sealing element are typically only selectively connected. They are not fixedly bound to one another along the entire length of the hose, as is the case in existing hose designs. Thus, the structural element is free to move within or around the sealing element. Such a design can provide an enhanced user experience compared to that of existing hose designs, due, at least in part, to increased flexibility, more desirable torque response, and easier transportability.

The structural element can define a longitudinally extending volume and comprise a plurality of interconnected subelements. The interconnected subelements can be movable with respect to one another to bend the volume defined by the structural element. The subelements either separate or come together upon bending of the hose. The subelements positioned on the inside of the bend will come together while the subelements positioned on the outside of the bend will separate. An example of a structural element includes a helical element in which the subelements comprise winds of the helix. Other embodiments are also possible. For example the structural element can comprise a plurality of circumferential rings connected longitudinally along the length of the hose. Embodiments of structural elements are described in more detail below.

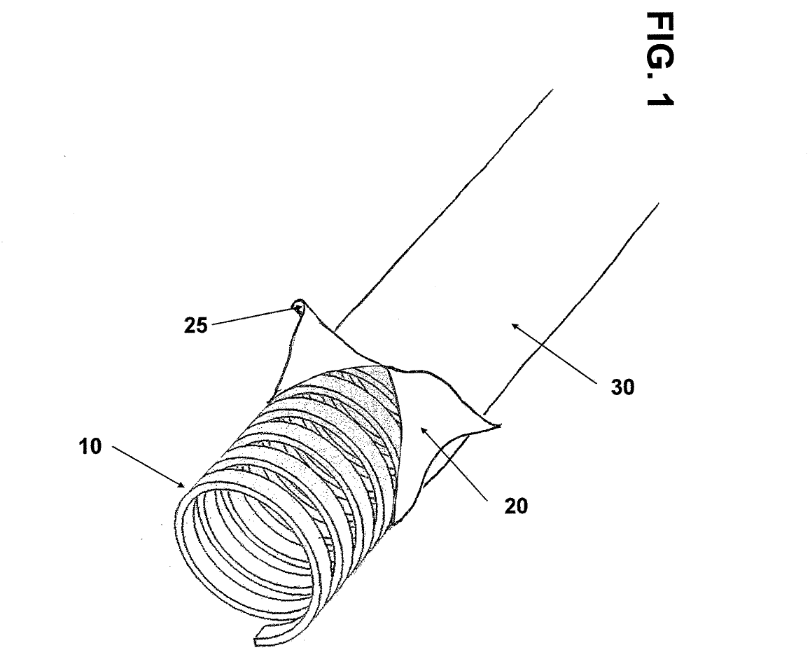

FIG. 1 shows a perspective view of the tubing or hose 5, comprising a structural element 10 with a surrounding outer layer 30. In FIG. 1, the outer layer 30 is peeled back for illustrative purposes. The structural element 10 is shown with a spiral construction. The outer or sealing layer 30 is shown surrounding the structural element 10. The sealing layer can prevent the loss of gas from the tubing. In some embodiments, the sealing layer comprises multiple layers and/or materials, for example an inner layer 20 and an outer layer 25, as shown in FIG. 1. In some embodiments, the inner layer 20 provides the gas impermeable characteristics, and the outer layer 25 provides the cosmetic and durable characteristics. Other configurations are also possible. For example, a single layer can provide gas impermeable and cosmetic and durable characteristics.

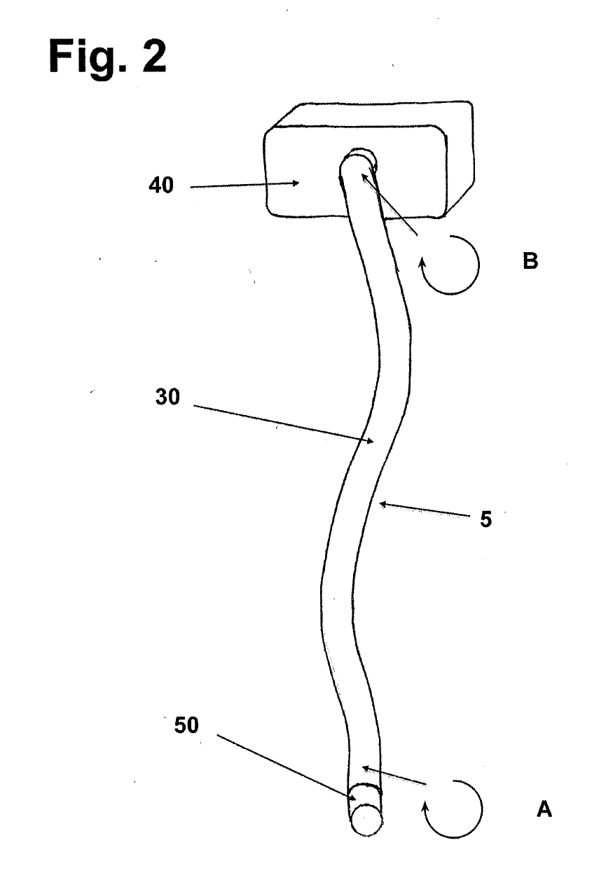

The tubing disclosed herein can provide a number of advantages over the prior art. The tubing can provide better flexibility than existing designs. Increased flexibility can, for example, allow the hose to move with a user, experience fewer mask leaks and less tugging on the hose, and be easier to pack for transport. The tubing can exhibit enhanced torque characteristics (e.g., be torsion-absorbing), allowing for more comfortable use and less tugging on the mask or the device end of the tube. This property is described in more detail below, with respect to FIG. 2 and Table 1. The hoses described herein can be more cosmetically fit for a bedroom environment than existing hoses. The hoses can have a less distinctly clinical or medical look, better blending in with bedroom decor. The hoses of the present application can utilize less bulky connectors having a lower profile and lower weight than existing connectors. The hoses can have an outer layer (e.g., impermeable layer or cover) without the ridges of the prior art hoses. A smooth outer layer can allow the hoses to be quieter during use as there are no ridges to rub against surrounding surfaces.

The hoses can also comprise a lighter weight overall, allowing for more comfortable use and easier travel. For example, the hoses can exhibit up to 80% weight reduction over existing hoses through proper design and materials choice. The tubing described herein can weigh about 35-55 g, in some embodiments. This compares very favorably to the current tubing on the market, which weighs 55-175 g per tube. Thus the tubing described herein offers the possibility of up to 80% weight reduction. Weight reduction reduces drag on the tubing, pulling on the mask, and allows for great freedom of movement. It can also be easier to pack and transport for travel.

FIG. 2 shows the tubing 5 connected to a positive airway pressure device 40. The connector 50 is shown at one end of the tubing. The rotational arrows at A and B illustrate the experimental setup and method used to determine the torsion of the tube when a torque is applied. These results are shown in Table 1, below.

The tubing disclosed herein can provide enhanced torque behavior compared to existing designs, as described above. The tubing can exhibit about 3:1-6:1 torque characteristics, meaning the tubing is turned about 3-6 times at one end before the opposite end rotates. This capability provides a significant comfort advantage for the user. It means the tube will not resist the user's movements in the same way. It will adapt to their movement 3-6 times better than a standard tube.

TABLE 1
Torsion characteristics

| | | Number of degrees of | | | |
|---|---|---|---|---|---|
| | | rotation applied at A to | | | Normal- |
| | | result in movement at B | | Minimum | ized to |
| Tube | Description | Clockwise | Counter CW | per tube | baseline |
| Prior Art | 4 ft. length, | 90 | 180 | 90 | 1x |
| Tube | 15 mm | | | | |
| | diameter | | | | |
| Prototype1 | 4 ft. length, | 2520 | 1080 | 1080 | 12x |
| | 15 mm | | | | |
| | diameter, | | | | |
| | materials 1 | | | | |
| Prototype2 | 4 ft. length, | 630 | 1080 | 630 | 7x |
| | 15 mm | | | | |
| | diameter, | | | | |
| | materials 2 | | | | |
| Prototype3 | 4 ft. length, | 450 | 540 | 450 | 5x |
| | 22 mm | | | | |
| | diameter, | | | | |
| | materials 2 | | | | |

Table 1 shows the results from testing of various tubes versus the prior art standard tubing. When torsion results are normalized to the prior art tubing, the flexible tubing allows about 5-25 times more turns before transferring that torsional force to the distant end of the tubing. Compared to existing tubes, the flexible tubing disclosed herein yields a more comfortable, more adaptable tubing which does not oppose the users' movements.

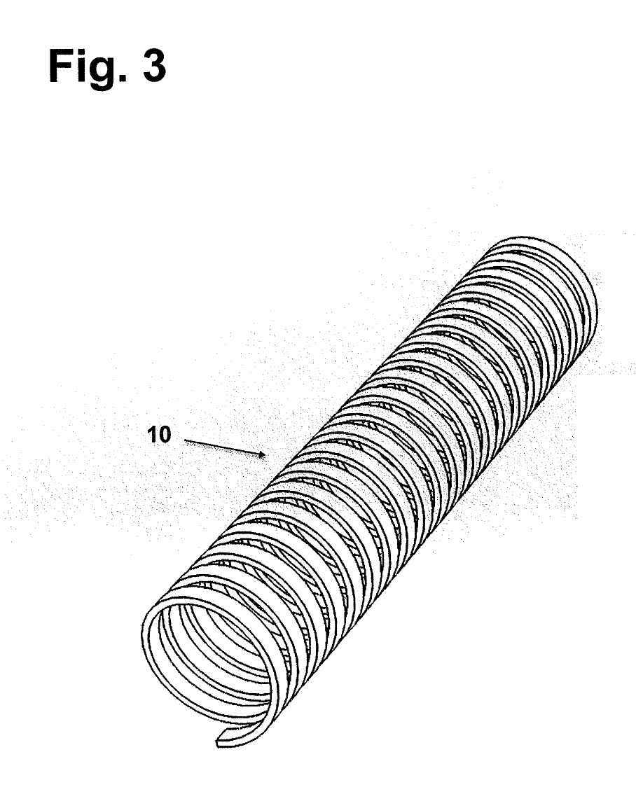

FIG. 3 illustrates the structural element in a coil, spiral, or helical construction. The winds of the coil can have a typical width of about 1-5 mm. In some cases, the winds of the coil may have a larger width, in the range of 5-10 mm or larger. Winds of the coil (e.g., adjacent winds) are shown as having the same thickness, but wind thickness can vary, in some embodiments. Coil pitch or the distance from the center of one coil to the center of the adjacent coil, can be typically in the range of about 1-10 mm. In some cases, the pitch of the coil may be larger, in the range of about 10-15 mm. Distance between adjacent winds can be generally uniform, as shown in FIG. 3, or can vary. The thickness of the structural element can typically be about 0.5-2.0 mm. In some cases, the thickness of the structural element can be about 0.25-4 mm. The thickness can be uniform along the length of the structural element, or can vary.

While FIG. 1 shows an embodiment of the tube 5 with a sealing layer 30 on the outside of the tube, in some embodiments, the sealing layer can also be on the inside of the structural element. FIG. 4 illustrates an embodiment of the tube 5 with the sealing or gas impermeable liner 60 on the inside of the spiral structural element 10. The illustration shows the gas impermeable liner 60 peeled back to show the inner surface 61 and the outer surface 62.

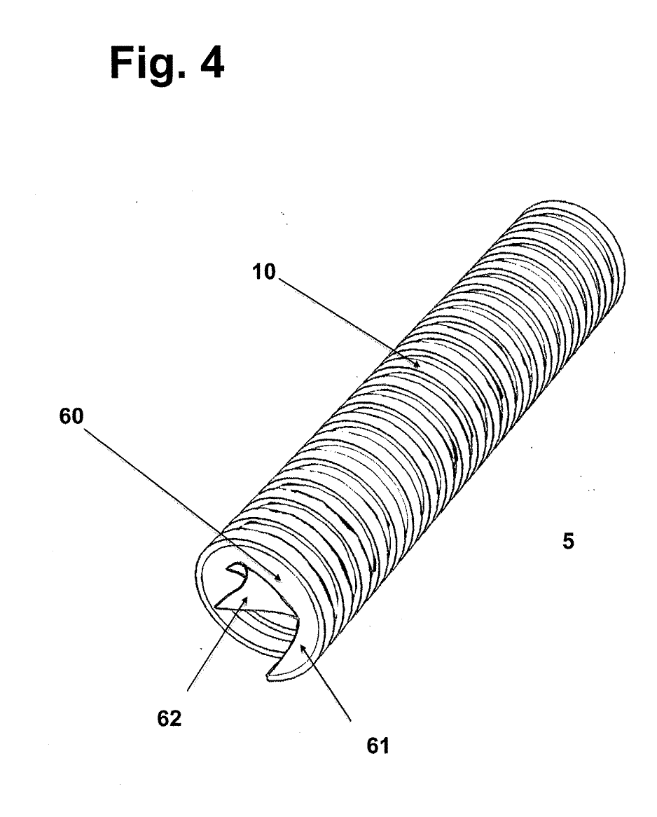

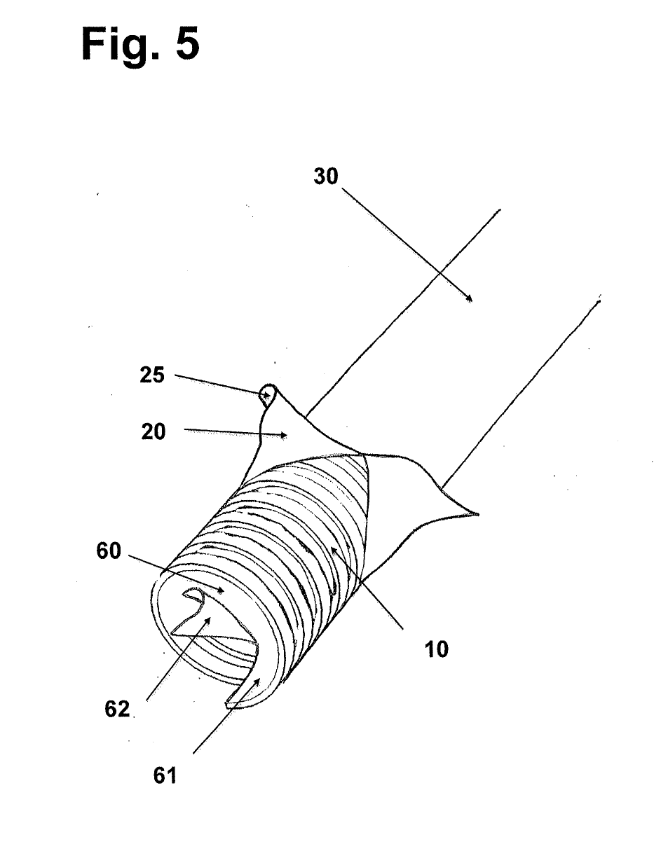

FIG. 5 illustrates an embodiment of the tube 5 with the gas impermeable layer 60 on the inside of the spiral structural element 10. The illustration shows the gas impermeable liner 60 peeled back to show the inner surface 61 and the outer surface 62. Shown also is the outer layer 30. In this embodiment, the outer layer 30 is cosmetic and durable. It can be made from materials which are soft and appealing to fit the bedroom use environment. The outer layer 30 is peeled back to show its inner surface 20 and outer surface 25.

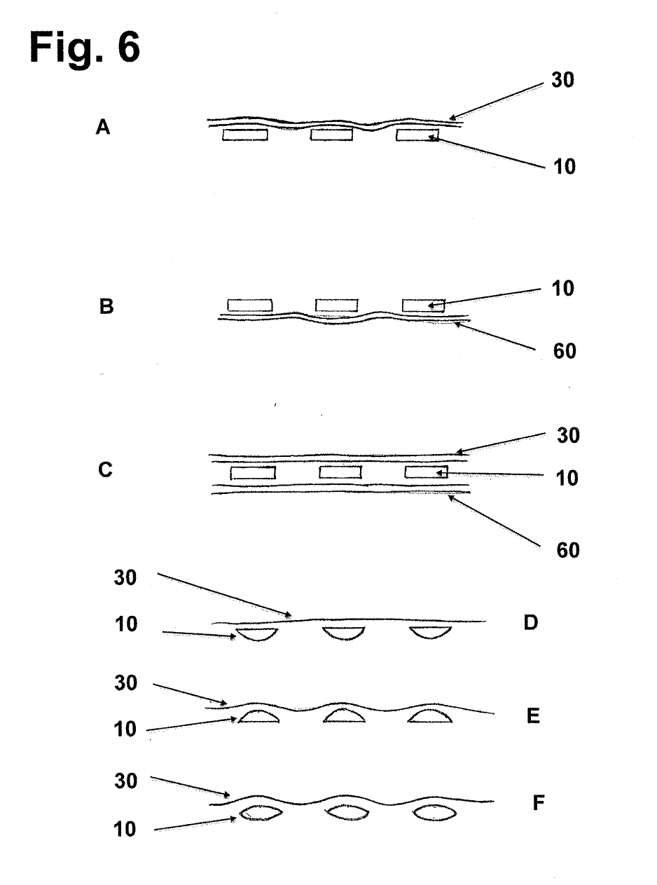

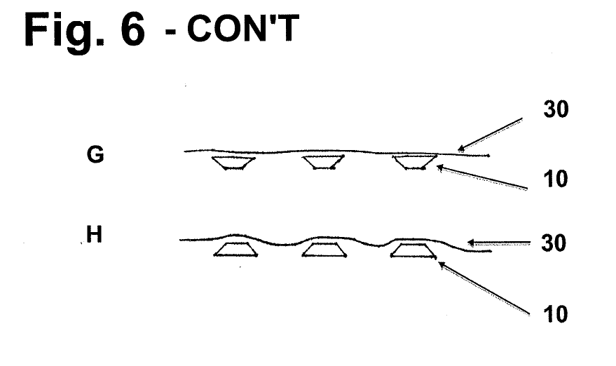

FIGS. 6A-F illustrate several embodiments of construction, presented in longitudinal single wall cross section. FIG. 6A shows the spiral structural element 10, with the gas impermeable layer 30 on the outer surface. FIG. 6B shows the spiral structural element 10, with the gas impermeable layer 60 on the inner surface. FIG. 6C shows the spiral structural element 10, with the layer 30 on the outer surface, and the gas impermeable layer 60 on the inner surface.

FIGS. 6D, 6E, 6F, 6G, 6H all show various cross sectional geometries of the spiral structural element 10. In FIG. 6D, the innermost surface of the element 10 is rounded and the outermost surface is flat. In FIG. 6E, the innermost surface of the element 10 is flat and the outermost surface is rounded. In FIG. 6F, both the innermost surface and the outermost surface of the element 10 are rounded. In FIG. 6G, the innermost surface is shorter than the outermost surface, providing increased surface area for contact with the impermeable layer 30, and a smoothed inner path for airflow. In FIG. 6H, the innermost surface is longer than the outermost surface, providing increased flexibility at the interface with the impermeable layer 30. In various constructions, the inner surface can be rounded to enhance airflow. In other embodiments, a flat inner surface aids in construction of the structural element.

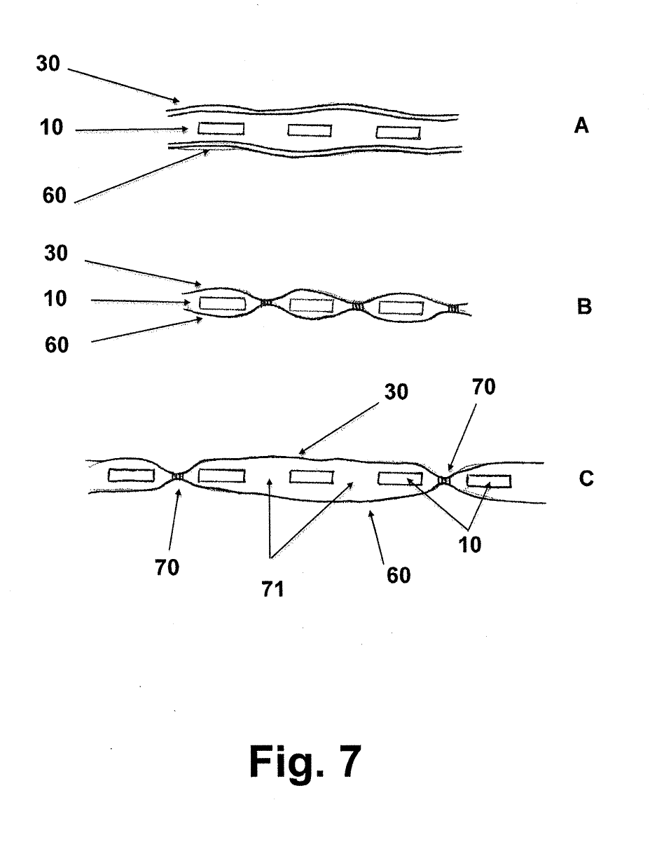

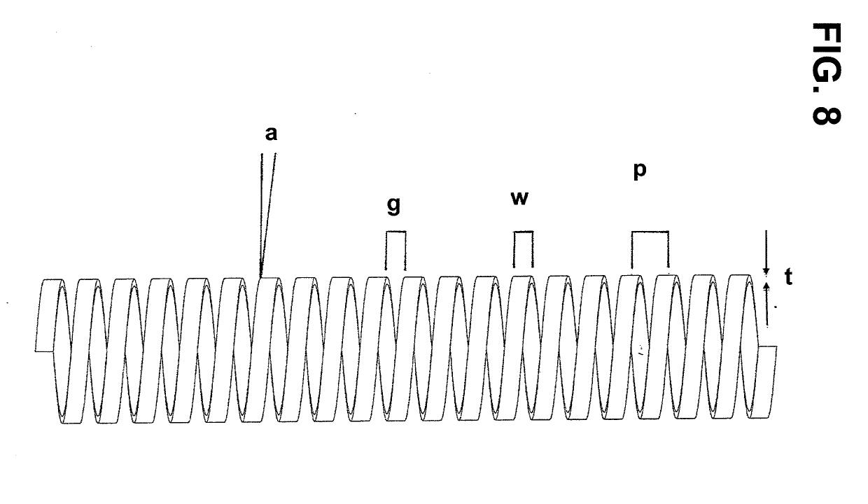

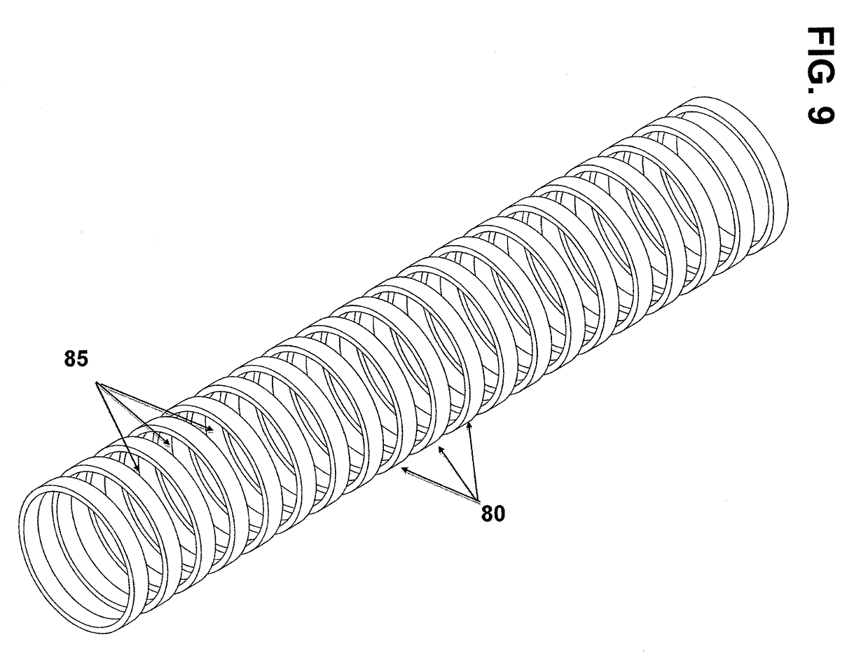

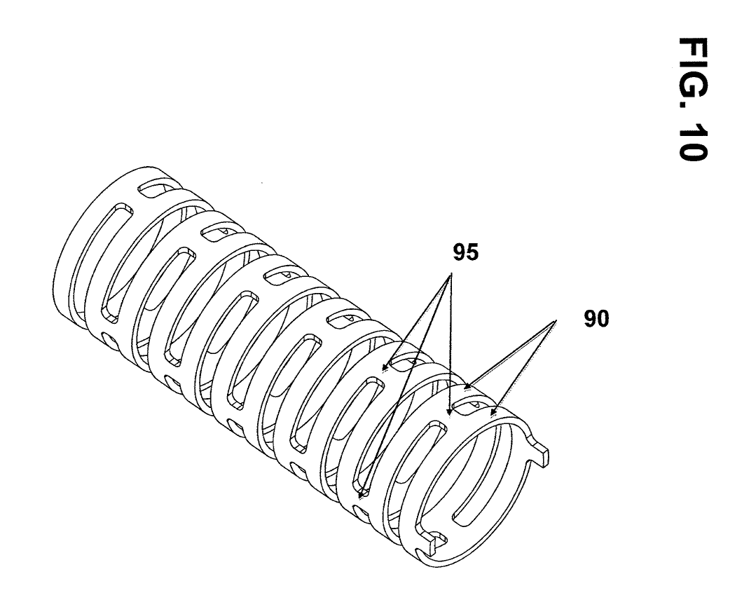

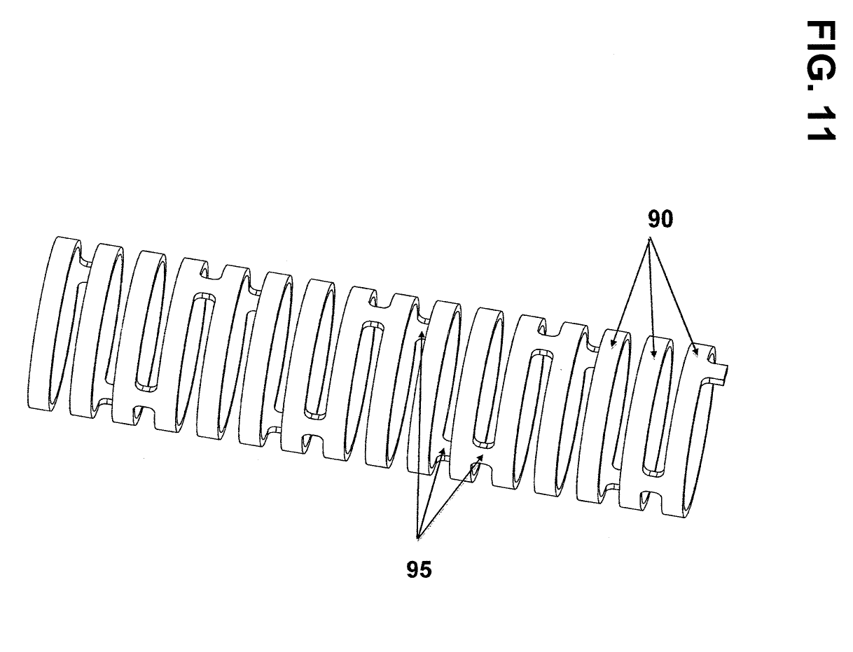

FIGS. 7A, 7B, and 7C show various construction techniques for the tubing. In FIG. 7A, the inner layer 60 and outer layer 30 are not connected to each other, nor are they attached to the structural element 10 along the majority of the tube. The layers may be attached to the structural element at each end of the tubing.

In FIG. 7B, the inner layer 60 and outer layer 30 are connected to each other between the structural element 10, but they are not attached to the structural element 10.

In FIG. 7C, the inner layer 60 and outer layer 30 are connected to each other, but not at each opportunity. They are connected periodically. A connection is shown every third space in this figure; however, many other combinations of skipping and connecting are possible. For example, a connection can be made every other space or every four spaces.

In FIG. 8, the pitch, p, is shown as the distance from the center of one coil to the center of the adjacent coil. The coil width, w, is also shown. The gap, g, is shown to be the space between two adjacent coils, and can typically be in the range of about 2-7 mm. In some cases the gap may be larger, in the range of about 10-15 mm. The pitch angle, a, is shown as the angle degree of the coil pitch, and can typically be in the range of about 2-10 degrees. In some cases, the pitch angle may be larger, in the range of 15-50 degrees. Each of these terms, separately or in combination, can be used to describe the construction of the structural element.

While the preceding embodiments included helical structural elements and winds as subelements, other structural elements and subelement designs are also possible. In FIG. 9, a structural element is shown which is comprised of a series of circumferential rings 80. These rings 80 provide radial support against collapse, while the spaces 85 between them allow for flexibility. This structural element can be combined with the impermeable layer described herein. Each circumferential ring can be at least partially connected to the impermeable layer, such that the assembly maintains the overall structure of the hose.

In FIG. 10, a structural element is shown, comprising radial ring elements 90 connected to the adjacent radial element 90 in two places each. The connections 95 can be arranged advantageously around the circumference of the tubing.

In FIG. 11, a structural element is shown, comprising radial ring elements 90 connected to the adjacent radial element 90 in one place. The connections 95 can be arranged advantageously around the circumference of the tubing.

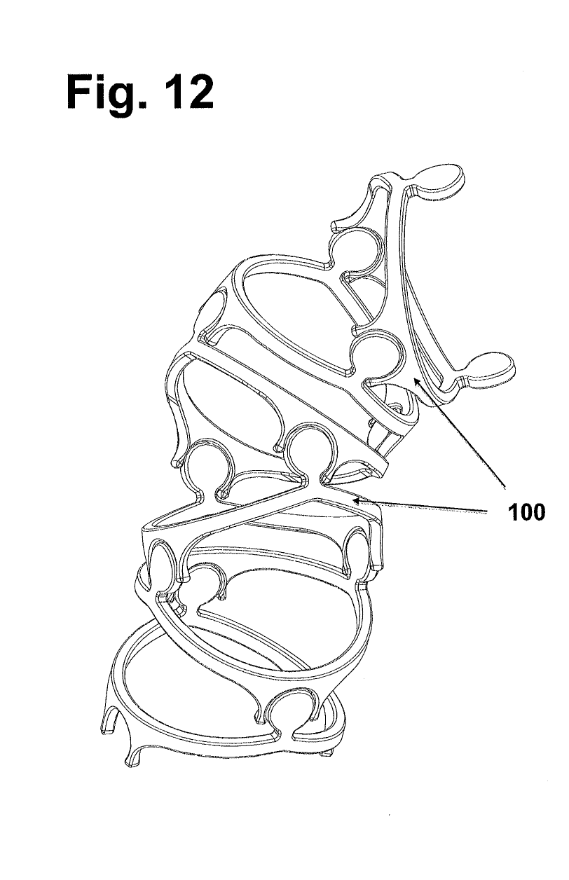

In FIG. 12, a structural element is shown, comprising multiple units of interlocking structural radial elements 100 which allow for excellent flexibility while also maintaining radial strength. The assembly in FIG. 12 shows two complex curves to illustrate the flexibility of the structure.

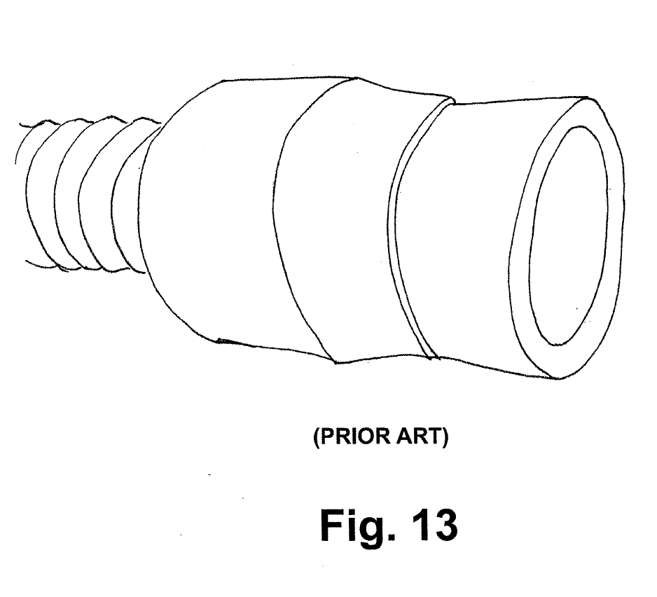

In FIG. 13, a tubing connector from the prior art is shown. It can be seen that it is much bulkier in diameter than the tubing, incorporating excess size and weight.

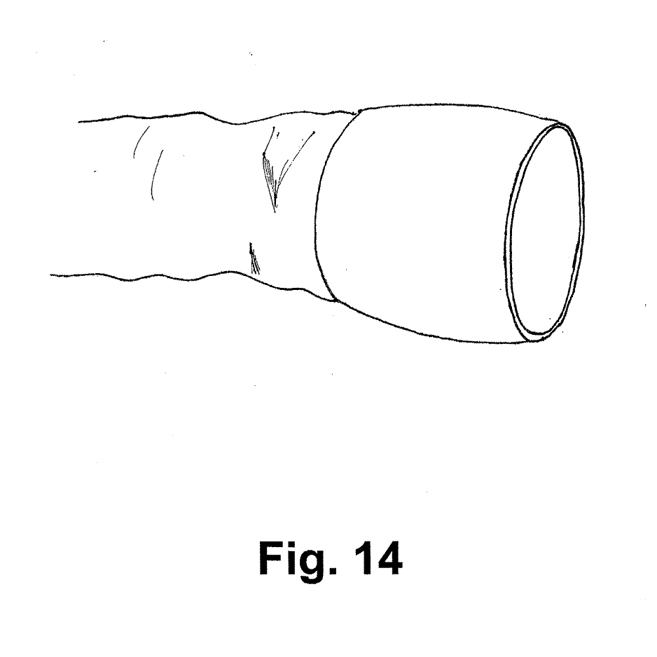

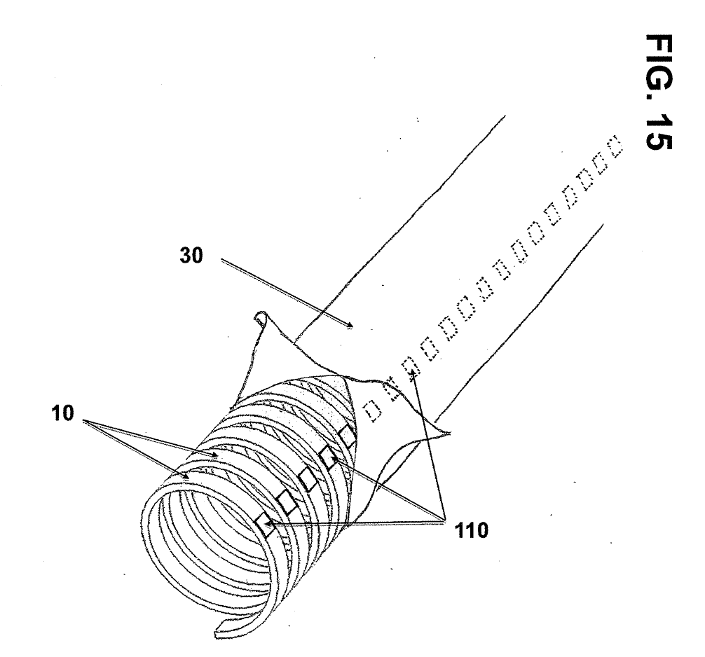

In FIG. 14, a tubing connector is shown with a minimal diameter change relative to the diameter of the tubing. This connector is lightweight, low profile, and integrates more completely with the tubing. It can be constructed of rigid, semi-rigid, or flexible plastic or elastomeric materials. It can be sized to fit standard tubing fitting sizes common in the industry, such as 22 mm fittings.

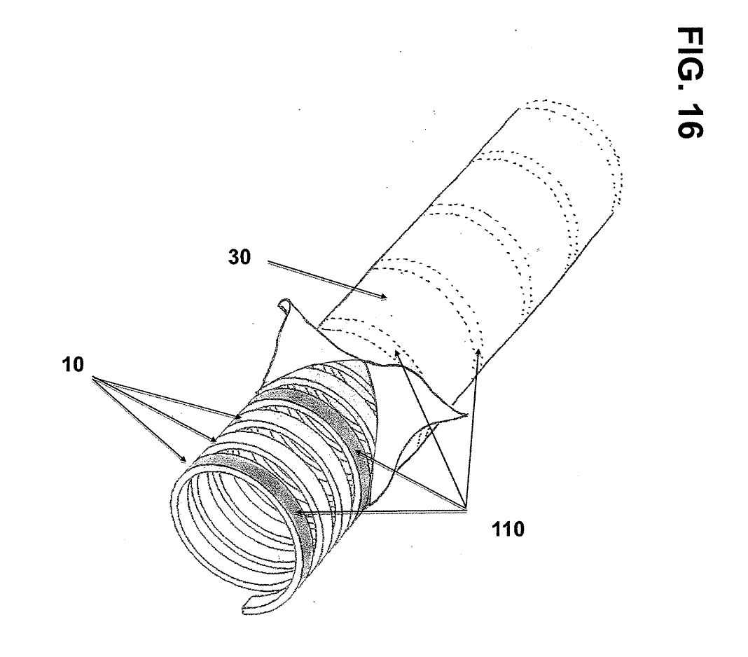

In FIG. 15, the structural element 10 and the impermeable layer 30 are shown, with a pattern of attachment zones 110 illustrated lengthwise along the tube assembly. These attachment zones are shown on each coil of the structure, but they could alternate coils or be selectively placed intermittently on the coils. Additionally, although only one row of attachment zones is shown, there could be multiple such rows along the length of the tubing. This selective connection between the structural element and the impermeable layer allows for great flexibility.

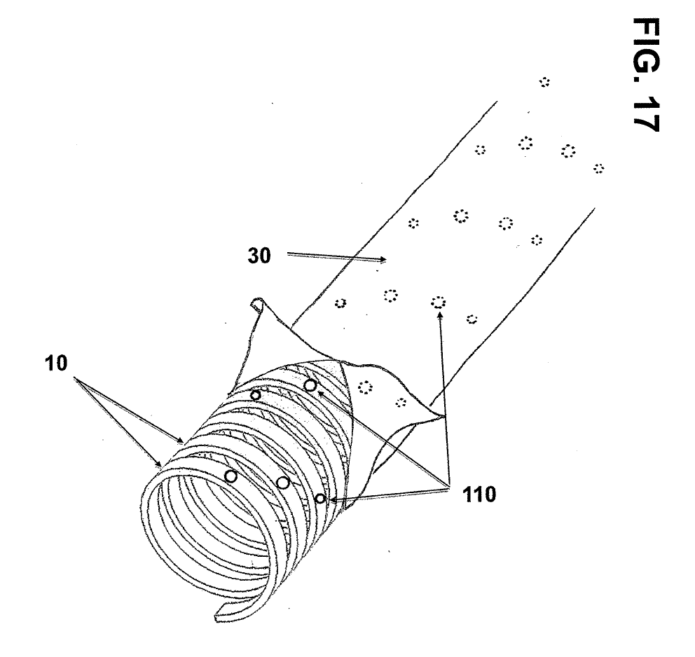

In FIG. 16, the structural element 10 and the impermeable layer 30 are shown, with a pattern of attachment zones 110 illustrated along the tube assembly. These attachment zones are shown on intermittent coils of the structure, but they could be selectively placed in a different pattern on the coils. FIG. 16 shows the attachment zones on every third coil, however, this pattern could vary considerably, with the attachment zones on every other coil, or only on every 20 coils or more. This selective connection between the structural element and the impermeable layer allows for great flexibility.

In FIG. 17, the structural element 10 and the impermeable layer 30 are shown, with a pattern of attachment zones 110 illustrated along the tube assembly. These attachment zones are shown on each coil of the structure in a radially rotated placement. Variations of the illustrated pattern are possible, including multiple attachment zones on each coil, and more intermittent patterns. This selective connection between the structural element and the impermeable layer allows for great flexibility.

The extent of connection between the structural element and the impermeable layer can be varied as shown in and described with respect to FIGS. 7A-C and 15-17, above. Fewer connections between the structural element and the impermeable layer will increase the overall flexibility of the hose. For example, a hose comprising a structural element not connected to the impermeable layer at all, or only at the ends will be more flexible than a similar hose comprising a structural element intermittently connected to the impermeable layer. However, even those hoses comprising structural elements intermittently connected to the impermeable layer will exhibit greater flexibility than existing hoses in which the impermeable layer and structural elements are connected along the length of the hose.

The tubing assemblies described herein allow for greater flexibility and less torsional resistance. But another advantage they have is that they exhibit significantly less memory than traditional tubes of the prior art. Prior art tubes are typically made of one or two thermoset plastics, fully integrated along the entire length of the tube, so that they essentially become one structure. One limitation of this approach of the prior art tubes is that the tubing tends to take a set, or exhibit material memory, when placed in one position for an amount of time. This makes the tubing more difficult to comfortably use for the user. The tubing tends to want to assume the position of its set, which most often is different from the preferred position for the user.

An advantage of the tubing assemblies described herein is that they do not take a set, or exhibit material memory. This allows these tubing assemblies to be more flexible, and therefore more comfortable for the user.

In some embodiments, the structural element is free of connection to the sealing element over a majority of the hose. The structural element can be free of connection to the sealing element over about 90% or more of the length of the hose. Other configurations are also possible. For example, the structural element can be free of connection to the sealing element over about 25%, 40%, 50%, 80%, 90, or 95% of the length of the hose.

The sealing element can comprise any material that is both flexible and largely impermeable to gases. This includes but is not limited to cloth, textiles, woven materials, fabric, plastics, woven plastics, composites, laminates.

The structural element can comprise plastics, elastomers, metals, metals with memory characteristics, wood, paper, adhesives, cloth, fabric, stitching, woven materials, and composites such as metal and plastic. Additionally, the structural element may include a heating element. In one embodiment, the heating element can be comprised of a metal wire with desired resistance to provide heating to the tubing interior.

In some embodiments, the hose can comprise a heating element to maintain the inner temperature above the dew point of air transported within the hose, prevented condensation or rain out. The heating element can comprise a wire than runs the length of the hose. The wire can be connected to the structural element, sealing element, or the cover. In some embodiments, the wire can be part of a separate structure used in conjunction with the hose. Examples of such heating elements are described in U.S. Publication No. 2013/0333701, filed Jun. 18, 2012, U.S. Pat. No. 8,733,349, filed Jul. 30, 2010, and U.S. Pat. No. 6,918,389, filed Mar.14, 2001, the disclosures of each of which are incorporated by reference herein.

It will be appreciated that the terms spiral, helix, coil can all be used to describe the structural element geometry.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-54. (canceled)

55. A hose for use with a respiratory device, the hose comprising:
a patient end comprising a mask connector having a port adapted to communicate with a breathing mask;
an air source end comprising an air source connector having a port adapted to communicate with an air source;
a structural element connected to the mask connector and to the air source connector; and
a sleeve extending from the mask connector to the air source connector to provide an airtight lumen between the mask connector port and the air source connector port, the sleeve being free of connection to the structural element over at least a portion of the length of the sleeve.

56. The hose of claim 55, wherein the structural element and sleeve are only connected at ends of the structural element and sleeve.

57. The hose of claim 55, wherein the sleeve is positioned within the structural element.

58. The hose of claim 55, wherein the sleeve covers the structural element.

59. The hose of claim 55, further comprising a gas impermeable liner positioned within the structural element.

60. The hose of claim 59, wherein the sleeve and the liner are connected to each other between portions of the structural element.

61. The hose of claim 55, wherein the sleeve is gas impermeable.

62. The hose of claim 55, wherein the structural element comprises a helical element.

63. The hose of claim 55, wherein the sleeve is free of connection to the structural element over at least 50% of the length of the sleeve.

64. A hose for use with a respiratory device, the hose comprising:
a patient end comprising a mask connector having a port adapted to communicate with a breathing mask;
an air source end comprising an air source connector having a port adapted to communicate with an air source;
a structural element extending between the mask connector and to the air source connector, the structural element defining a longitudinally extending volume, the structural element comprising a plurality of interconnected subelements that are movable with respect to each other to bend the volume; and
a sleeve extending from the mask connector to the air source connector to provide an airtight lumen between the mask connector port and the air source connector port, the sleeve being free of connection to the structural element between at least two adjacent subelements.

65. The hose of claim 64, wherein the structural element comprises a helix.

66. The hose of claim 64, wherein the subelements comprise at least one of radial rings and interlocking structural radial elements.

67. The hose of claim 64, wherein the at least two adjacent subelements are connected to the sleeve.

68. The hose of claim 64, wherein the structural element is connected to the sleeve on either side of the at least two subelements.

69. The hose of claim 64, wherein the structural element is connected to the sleeve at the ends of the hose.

70. The hose of claim 64, wherein the structural element is free of connection to the sleeve along an entire length of the hose.

71. A method of manufacturing a hose for use with a respiratory device, the method comprising:
providing a structural element comprising a patient end having a mask connector having a port adapted to communicate with a breathing mask, the structural element further comprising an air source end having an air source connector having a port adapted to communicate with an air source;
providing a sleeve extending from the mask connector to the air source connector to provide an airtight lumen between the mask connector port and the air source connector port; and
coupling the sleeve and the structural element such that the sleeve is free of connection to the structural element over at least a portion of the length of the sleeve.

72. The method of claim 71, wherein the structural element comprises a plurality of interconnected subelements that are movable with respect to each other to bend the volume, and wherein coupling the sleeve comprises coupling the sleeve and structural element such that the sleeve is free of connection to the structural element between at least two adjacent subelements.

73. The method of claim 71, wherein providing the sleeve comprises inserting the sleeve within the structural element.

74. The method of claim 71, wherein providing the sleeve comprises covering the structural element with the sleeve.
